(19) United States
(12) Patent Application Publication
(10) Pub. No.: US 2026/0068009 A1
(43) Pub. Date: Mar. 5, 2026

(54) MYOPIA PREVENTION LIGHTING APPARATUS AND ITS CONTROL METHOD

(71) Applicant: THE CATHOLIC UNIVERSITY OF KOREA INDUSTRY-ACADEMIC COOPERATION FOUNDATION, Seoul (KR)

(72) Inventor: Dong Hyun JEE, Seoul (KR)

(73) Assignee: THE CATHOLIC UNIVERSITY OF KOREA INDUSTRY-ACADEMIC COOPERATION FOUNDATION, Seoul (KR)

(21) Appl. No.: 19/319,706

(22) Filed: Sep. 4, 2025

(30) Foreign Application Priority Data
Sep. 5, 2024 (KR) 10-2024-0120972

(51) Int. Cl. H05B 45/22; A61N 5/06; H05B 47/16

(52) U.S. Cl. CPC H05B 45/22; A61N 5/0613; H05B 47/16; A61N 2005/0651; A61N 2005/0663

(57) ABSTRACT

Disclosed are a myopia prevention lighting apparatus and a control method thereof, capable of enhancing the circadian rhythm by adjusting the illuminance of a lighting unit. A myopia prevention lighting apparatus according to an aspect of the present disclosure may comprise an LED lighting unit including a white LED that emits white light, a blue LED that emits blue light, and a red LED that emits red light having a wavelength range longer than that of the blue LED; a power supply unit configured to supply power to the LED lighting unit; a timer configured to measure time in order to determine day and night cycles; a light quantity adjustment unit configured to adjust the amounts of the white light, the blue light, and the red light by regulating power supplied from the power supply unit; and a control unit configured to control the light quantity adjustment unit according to a programmed setting such that the mixing ratios of the white light, the blue light, and the red light are adjusted over time to be equal to or greater than 5,000 lux for a certain period during the daytime and equal to or less than 500 lux for a certain period during the nighttime.

### CROSS-REFERENCE TO RELATED APPLICATION

This application claims priority to and the benefit of Korean Patent Application No. 10-2024-0120972, filed on Sep. 5, 2024, the disclosure of which is incorporated herein by reference in its entirety.

### BACKGROUND

### 1. Technical Field

The present disclosure relates to a lighting apparatus, and more particularly, to a myopia prevention lighting apparatus and a control method thereof capable of enhancing the circadian rhythm by adjusting the illuminance.

### 2. Related Art

In general, myopia is an ophthalmic condition in which only nearby objects are seen clearly, and it typically develops and progresses during childhood and adolescence. When myopia occurs, eyeglasses or contact lenses are required, and in severe cases, it may lead to serious complications such as glaucoma, macular degeneration, or retinal detachment. In recent years, this country has seen a rapid increase in the prevalence of myopia among adolescents, which has become a growing public health concern.

There are various causes of myopia, but recent studies have identified insufficient exposure of the eyes to sunlight during the daytime in childhood and adolescence as the most significant cause. Specifically, the retina, a neural tissue in the eye, must be sufficiently exposed to light during the day for the hormone dopamine to be secreted in the retina, which helps prevent myopia. In particular, intrinsic photosensitive cells in the retina are responsible for this function.

Subsequent studies have revealed that adequate exposure to light during the day and, conversely, reduced exposure to light at night are necessary.

However, in modern society, students are not sufficiently exposed to light on their retinal cells during the day. As a result, the circadian rhythm—where an adequate amount of dopamine is secreted during the day and a sufficient amount of melatonin is secreted at night—is disrupted, leading to the development of myopia.

The required light intensity during the day is 5,000 lux, and the light intensity at night must be 500 lux or less in order to activate the circadian rhythm and prevent the development of myopia.

Conventional lighting units (i.e., lighting fixtures including, but not limited to, LEDs and other types of light sources along with optical components) emit light at a constant intensity regardless of whether it is day or night. As a result, the lighting used by adolescents in this country—who spend more time studying indoors than their peers in any other country—has been negatively affecting their vision and contributing to the development of myopia. However, it is not realistic for students to stop studying and instead play outside or study under natural sunlight.

### SUMMARY

The present disclosure is to solve the above-described problems, and the present disclosure is directed to providing a myopia prevention lighting apparatus and a control method thereof, which are capable of preventing the onset of myopia or suppressing the progression of existing myopia by irradiating bright light of 5,000 lux during the daytime and dim light of 500 lux during the nighttime according to time.

The problems of the present disclosure are not limited to those mentioned above, and other problems not mentioned will be clearly understood by those of ordinary skill in the art from the following description.

According to an aspect of the present disclosure, provided is a myopia prevention lighting apparatus comprising an LED lighting unit including a white LED that emits white light, a blue LED that emits blue light, and a red LED that emits red light having a wavelength range longer than that of the blue LED; a power supply unit configured to supply power to the LED lighting unit; a timer configured to measure time in order to determine day and night cycles; a light quantity adjustment unit configured to adjust the amounts of the white light, the blue light, and the red light by regulating power supplied from the power supply unit; and a control unit configured to control the light quantity adjustment unit according to a programmed setting such that the mixing ratios of the white light, the blue light, and the red light are adjusted over time to be equal to or greater than a first illuminance for a certain period during the daytime and equal to or less than a second illuminance for a certain period during the nighttime.

In this case, the first illuminance may be in a range of 5,000 to 15,000 lux.

In this case, the second illuminance may be in a range of 0 to 500 lux.

In this case, the total mixing ratio of the blue light and the red light may be constant over time, the mixing ratio of the blue light may be higher during the daytime, and the mixing ratio of the red light may be higher during the nighttime.

In this case, the total mixing ratio of the blue light and the red light may be equal to the mixing ratio of the white light.

In this case, the control unit may be configured to reduce a maximum variation of the blue light if the amount of light detected at a position irradiated by the LED lighting unit is greater than a threshold value, and to increase the maximum variation of the blue light if the detected light amount is equal to or less than the threshold value.

In this case, the threshold value may be 2,500 lux.

In this case, the control unit may be configured to control the total mixing ratio of the blue light and the red light to be lower than the mixing ratio of the white light in the case where natural lighting is present or in the case of local-area lighting; and control the total mixing ratio of the blue light and the red light to be higher than the mixing ratio of the white light in the case where natural lighting is absent, in the case of full-area lighting, or in the case of a cataract patient.

According to another aspect of the present disclosure, a control method for a myopia prevention lighting apparatus may comprise retrieving a setting of an LED lighting unit including a white LED that emits white light, a blue LED that emits blue light, and a red LED that emits red light having a wavelength range longer than that of the blue light; adjusting mixing ratios of the white light, the blue light, and the red light over time according to a preprogrammed setting such that the illuminance is equal to or greater than a first illuminance for a certain period during the daytime and equal to or less than a second illuminance for a certain period during the nighttime; and turning on the LED lighting unit according to the adjusted mixing ratios.

In this case, the first illuminance may be in a range of 5,000 to 15,000 lux.

In this case, the second illuminance may be in a range of 0 to 500 lux.

In this case, the total mixing ratio of the blue light and the red light may be constant over time, the mixing ratio of the blue light may be higher during the daytime, and the mixing ratio of the red light may be higher during the nighttime.

In this case, the total mixing ratio of the blue light and the red light may be equal to the mixing ratio of the white light.

In this case, the control method for a myopia prevention lighting apparatus may include detecting an amount of blue light in the surroundings; comparing the detected amount of blue light with a threshold value; reducing a maximum variation of the blue light if the detected amount of blue light is greater than the threshold value; and increasing the maximum variation of the blue light if the detected amount of blue light is less than or equal to the threshold value.

In this case, the adjusting may include adjusting the total mixing ratio of the blue light and the red light to be lower than the mixing ratio of the white light in the case where natural lighting is present or in the case of local-area lighting; and adjusting the total mixing ratio of the blue light and the red light to be higher than the mixing ratio of the white light in the case where natural lighting is absent, in the case of full-area lighting, or in the case of a cataract condition.

According to the above configuration, the myopia prevention lighting apparatus and the control method thereof according to one aspect of the present disclosure can suppress the progression of myopia by increasing the amount of blue light during the daytime and decreasing the amount of blue light during the nighttime, thereby enabling effective management of eye health, such as myopia prevention, for adolescents in their growth period.

In addition, the myopia prevention lighting apparatus and the control method thereof according to an embodiment of the present disclosure can benefit not only adolescents but also adult users by enhancing their overall health. This is because the apparatus strengthens the circadian rhythm, which plays a vital role in maintaining human health-dopamine is secreted in the retina upon exposure to sunlight during the daytime, and melatonin is secreted during the nighttime in the absence of light. Recent studies have reported that disruption of the circadian rhythm is associated with increased prevalence of cardiovascular diseases and diabetes. Therefore, if the circadian rhythm is maintained in a healthy state, the health of modern individuals exposed to lighting can be improved.

Advantageous effects of the present disclosure are not limited to the above-described effects, and should be understood to include all effects that can be inferred from the configuration of the disclosure described in the detailed description or claims of the present disclosure.

### BRIEF DESCRIPTION OF THE DRAWINGS

FIG. 1 is a block diagram illustrating the configuration of a myopia prevention lighting apparatus according to an exemplary embodiment of the present disclosure.

FIG. 2 is a block diagram illustrating a program, which is one of the components of the myopia prevention lighting apparatus according to an exemplary embodiment of the present disclosure.

FIG. 3 is a flowchart illustrating a control method for the myopia prevention lighting apparatus according to an exemplary embodiment of the present disclosure.

FIG. 4 is a graph illustrating various cases in which only illuminance is controlled in the myopia prevention lighting apparatus according to an exemplary embodiment of the present disclosure.

FIG. 5 is a table showing an example of a basic mode of light control in the myopia prevention lighting apparatus according to an exemplary embodiment of the present disclosure.

FIG. 6 is a graph illustrating wavelength composition and changes in illuminance in the basic mode of the myopia prevention lighting apparatus according to an exemplary embodiment of the present disclosure.

FIG. 7 is a graph illustrating wavelength composition and changes in illuminance in the case of no natural lighting in the myopia prevention lighting apparatus according to an exemplary embodiment of the present disclosure.

FIG. 8 is a graph illustrating wavelength composition and changes in illuminance in the case of natural lighting in the myopia prevention lighting apparatus according to an exemplary embodiment of the present disclosure.

FIG. 9 is a graph illustrating wavelength composition and changes in illuminance in the case of local-area lighting in the myopia prevention lighting apparatus according to an exemplary embodiment of the present disclosure.

FIG. 10 is a graph illustrating wavelength composition and changes in illuminance in the case of full-area lighting in the myopia prevention lighting apparatus according to an exemplary embodiment of the present disclosure.

FIG. 11 is a graph illustrating wavelength composition and changes in illuminance in the case of a cataract patient using the myopia prevention lighting apparatus according to an exemplary embodiment of the present disclosure.

### DETAILED DESCRIPTION

Hereinafter, exemplary embodiments of the present disclosure will be described in detail so that those of ordinary skill in the art can readily implement the present disclosure with reference to the accompanying drawings. The present disclosure may be embodied in many different forms and is not limited to the embodiments set forth herein. In the drawings, parts unrelated to the description are omitted for clarity of description of the present disclosure, and throughout the specification, same or similar reference numerals denote same elements.

The words and terms used in the present specification and claims should not be interpreted as being limited to their ordinary or dictionary meanings, but should be construed as having meanings and concepts consistent with the technical spirit of the present disclosure, in accordance with the principle that an inventor may define terms and concepts to best describe their invention.

Accordingly, the embodiments described in the present specification and the configurations shown in the drawings correspond to preferred embodiments of the present disclosure, and do not represent all the technical spirit of the present disclosure, so the configurations may have various examples of equivalent and modification that can replace them at the time of filing the present disclosure.

It should be understood that the terms “comprise or include” or “have” or the like when used in this specification, are intended to describe the presence of stated features, numbers, steps, operations, elements, components and/or a combination thereof but not preclude the possibility of the presence or addition of one or more other features, numbers, steps, operations, elements, components, or a combination thereof.

The presence of an element in/on “front”, “rear”, “upper or above or top” or “lower or below or bottom” of another element includes not only being disposed in/on “front”, “rear”, “upper or above or top” or “lower or below or bottom” directly in contact with other elements, but also cases in which another element being disposed in the middle, unless otherwise specified. In addition, unless otherwise specified, that an element is “connected” to another element includes not only direct connection to each other but also indirect connection to each other.

Hereinafter, a myopia prevention lighting apparatus and a control method thereof according to an embodiment of the present disclosure will be described with reference to the drawings.

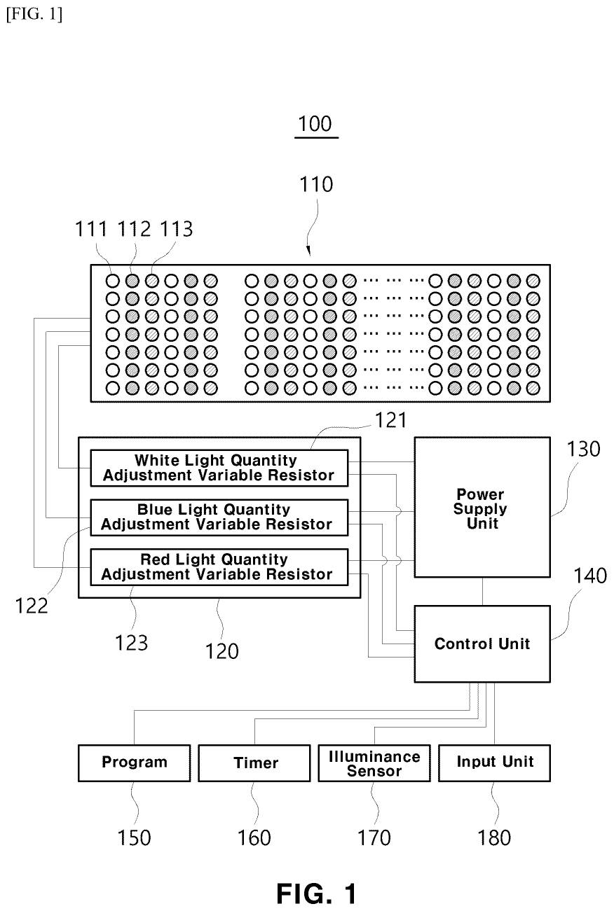

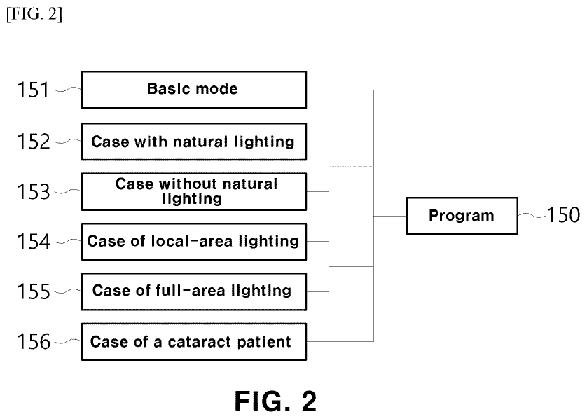

FIG. 1 is a block diagram illustrating the configuration of a myopia prevention lighting apparatus according to an exemplary embodiment of the present disclosure, and FIG. 2 is a block diagram illustrating a program, which is one of the components of the myopia prevention lighting apparatus according to an exemplary embodiment of the present disclosure.

Referring to FIG. 1, a myopia prevention lighting apparatus 100 according to an exemplary embodiment of the present disclosure includes an LED lighting unit 110, a light quantity adjustment unit 120, a power supply unit 130, a control unit 140, a program 150, and a timer 160.

The myopia prevention lighting apparatus 100 according to an exemplary embodiment of the present disclosure is intended to prevent myopia. For dopamine, a hormone, to be secreted in the retina-a neural tissue inside the eye—the retina must be sufficiently exposed to light during the day, which in turn helps prevent the development of myopia. In particular, an illuminance of 5,000 lux or higher, similar to that of natural sunlight, plays a key role in preventing myopia. Conversely, exposure to blue light in the evening can accelerate the progression of myopia. In summary, in order to prevent myopia, the retina must be exposed to blue light during the day and must not be exposed to blue light at night.

Accordingly, the myopia prevention lighting apparatus 100 is a lighting apparatus capable of adjusting both the wavelength composition and illuminance according to the day-night cycle. Specifically, the myopia prevention lighting apparatus 100 is designed to suppress the progression of myopia by emitting light that contains a higher proportion of blue light during the day and a lower proportion at night, while adjusting the illuminance based on the time of day. Through this configuration, the apparatus can effectively support eye health management, including the prevention of myopia, particularly for adolescents in their growth period.

In addition, the myopia prevention lighting apparatus 100 according to an exemplary embodiment of the present disclosure is intended to support effective health management. During the day, exposure to sunlight induces the secretion of dopamine in the retina, and at night, the absence of sunlight triggers the secretion of melatonin. This circadian rhythm is essential for maintaining human health.

Accordingly, the myopia prevention lighting apparatus 100 emits light containing blue light and red light depending on the time of day, thereby enhancing the circadian rhythm and promoting health while maintaining well-being during exposure to lighting.

As described above, the myopia prevention lighting apparatus 100 according to an embodiment of the present disclosure can contribute not only to the prevention of myopia in adolescents but also to the health of adults. Accordingly, it is possible to maintain or improve not only the eye health of the user but also their overall physical health.

The LED lighting unit 110 is an LED lighting means for providing a required amount of light during study and is configured such that the wavelength of the emitted light can be adjusted according to time. That is, the LED lighting unit 110 may provide white light, blue light, and red light in different ratios according to time. Through this, the illuminance of the provided light can be adjusted.

At this time, the LED lighting unit 110 includes a white LED 111, a blue LED 112, and a red LED 113. Here, the white LED 111 emits white light. The blue LED 112 emits blue light. The red LED 113 emits red light.

Here, the blue light may be light that promotes the secretion of dopamine. Accordingly, the blue light may be in the blue light wavelength range.

At this time, the blue light wavelength range may be 450 to 495 nm, and more preferably, 460 nm.

In addition, the light provided including the blue light may be provided at 5,000 lux or more. At this time, the appropriate illuminance of the blue light may be in the range of 5,000 to 15,000 lux.

In addition, the red light may be light that has a low melatonin-suppressing effect. Accordingly, the red light may be in the red light wavelength range. Preferably, the red light may be in the range of 630 to 780 nm, more preferably 660 nm, and the light provided including the red light may be 500 lux or less. At this time, the illuminance of the red light may be in the range of 0 to 500 lux.

That is, the LED lighting unit 110 may be composed of mixed light that emits white light for general illumination, blue wavelength ranges optimized for myopia prevention, and red wavelength ranges. The LED lighting unit 110 may emit white light, blue light, and red light according to the arrangement of LEDs for each wavelength.

The light quantity adjustment unit 120 adjusts the mixing ratio of the wavelengths of the light emitted from the LED lighting unit 110. At this time, the light quantity adjustment unit 120 adjusts the power supplied from the power supply unit 130 to control the light quantity of the white light, blue light, and red light. That is, the light quantity adjustment unit 120 can adjust the mixing ratio of the wavelengths by controlling the light quantity for each wavelength.

The light quantity adjustment unit 120 may include a white light quantity adjustment variable resistor 121, a blue light quantity adjustment variable resistor 122, and a red light quantity adjustment variable resistor 123.

The white light quantity adjustment variable resistor 121 adjusts the quantity of white light to provide an appropriate amount of illumination for reading by adolescents. The blue light quantity adjustment variable resistor 122 adjusts the blue light so that it is mainly emitted during daytime reading and reduced during nighttime reading. The red light quantity adjustment variable resistor 123 adjusts the red light so that it is mainly emitted during nighttime reading and reduced during daytime reading. Through this configuration, it is possible to provide a myopia suppression effect.

At this time, each of the white light quantity adjustment variable resistor 121, the blue light quantity adjustment variable resistor 122, and the red light quantity adjustment variable resistor 123 may adjust the amount of current supplied from the power supply unit 130 through the corresponding variable resistor for each wavelength range and supply the adjusted current to the LED lighting unit 110, thereby enabling adjustment of the light quantity of each wavelength in the LED lighting unit 110. Here, each of the white light quantity adjustment variable resistor 121, the blue light quantity adjustment variable resistor 122, and the red light quantity adjustment variable resistor 123 may be varied by the control unit 140.

The power supply unit 130 supplies power to the LED lighting unit 110. For example, the power supply unit 130 may be a switched-mode power supply (SMPS). The SMPS receives power from a power source and delivers it to the LED lighting unit 110 by modifying the current or voltage characteristics. Unlike a linear power supply, the pass transistor of the SMPS repeatedly switches on and off, oscillating between low-loss and high-loss points. As a result, power loss can be minimized since each state is maintained for only a very short period of time.

At this time, the SMPS can increase efficiency by raising the ON/OFF switching frequency with a fast cycle, thereby enabling the development of compact, lightweight, and high-capacity power supplies. In addition, while the SMPS has a wide input voltage range, it may generate ripple and noise due to heat generated by high-speed switching. Therefore, the ON/OFF timing of the output DC voltage is appropriately adjusted.

In addition, the power supply unit 130 may be a DC regulated power supply for providing a stable direct current (DC) power to the LED lighting unit 110, and may include a constant voltage circuit inside to suppress the generation of surge and noise.

The control unit 140 controls the overall operation of the myopia prevention lighting apparatus 100 and is configured to adjust the light quantity for each wavelength of the LED lighting unit 110 through the light quantity adjustment unit 120. That is, the control unit 140 controls the light quantity adjustment unit 120 to adjust the mixing ratio of the lights emitted from the LED lighting unit 110. Here, the control unit 140 may be a microcontroller. Accordingly, the change in the mixing ratio of the white light, blue light, and red light according to whether it is daytime or nighttime may be adjusted under the control of the control unit 140. Through this, light of 5,000 lux or more may be provided for a certain period of time during the day, and light of 500 lux or less may be provided at night.

At this time, the control unit 140 may control the ON/OFF switching of the power supply unit 130. In addition, the control unit 140 may control the white light quantity adjustment variable resistor 121, the blue light quantity adjustment variable resistor 122, and the red light quantity adjustment variable resistor 123 either simultaneously or individually, based on the settings of a myopia prevention program stored in the program 150 and according to the time measured by the timer 160.

At this time, the control unit 140 may adjust the optimal combination of light for myopia suppression at a preset ratio. That is, the control unit 140 may control the light quantity adjustment unit 120 so that the mixing ratio of white light, blue light, and red light is adjusted according to time based on preset settings.

At this time, the total mixing ratio of blue light and red light may remain constant over time. In addition, the mixing ratios of blue light and red light may be set according to the day-and-night circadian rhythm of the human body. For example, the mixing ratio of blue light may be higher during the daytime and may be highest around noon. The mixing ratio of red light may be higher during the nighttime and may be highest around midnight. At this time, light with the highest illuminance of 5,000 lux or more may be provided at noon, and light with the lowest illuminance of 500 lux or less may be provided at midnight.

In addition, the mixing ratios of white light, blue light, and red light may be adjusted according to the installation location of the myopia prevention lighting apparatus 100, the surrounding illuminance conditions, and the user's health condition such as eye-related diseases. For example, the installation location may be classified as either local-area lighting, such as a desk lamp, or full-area lighting installed on the ceiling. The illuminance condition may be classified based on the presence or absence of natural lighting and automatic setting according to the surrounding illuminance. The eye-related disease may be, for example, cataracts.

At this time, in the case where natural lighting is present or local-area lighting is used, the control unit 140 may control the total mixing ratio of blue light and red light to be lower than the mixing ratio of white light.

In addition, in the case of no natural lighting, full-area lighting, or cataracts, the control unit 140 may control the total mixing ratio of blue light and red light to be higher than the mixing ratio of white light.

Here, the total mixing ratio of blue light and red light may be highest in the case of cataracts and lowest in the case of natural lighting. Conversely, the mixing ratio of white light may be lowest in the case of cataracts and highest in the case of natural lighting.

In addition, the total mixing ratio of blue light and red light may be higher in the order of natural lighting absent, full-area lighting, and local-area lighting. Conversely, the mixing ratio of white light may be lower in the order of natural lighting absent, full-area lighting, and local-area lighting.

The program 150 stores settings according to a myopia prevention program. Here, the preset settings may be the time-based mixing ratios of white light, blue light, and red light based on the myopia prevention program. At this time, the program 150 may be integrally provided in a microcontroller together with the control unit 140.

Referring to FIG. 2, the program 150 may include settings based on the myopia prevention program, such as a basic mode 151, a case with natural lighting 152, a case without natural lighting 153, local-area lighting 154, full-area lighting 155, and a case of a cataract patient 156. Each of these settings will be described below with reference to FIGS. 5 to 11.

The timer 160 counts time. That is, the timer 160 may provide day and night cycle information to the control unit 140. At this time, the timer 160 may be integrally provided in a microcontroller together with the control unit 140.

Meanwhile, the myopia prevention lighting apparatus 100 may further include an illuminance sensor 170 and an input unit 180.

The illuminance sensor 170 may detect the ambient light quantity. At this time, the illuminance sensor 170 may detect the day and night cycle time based on the detected light quantity. For example, the illuminance sensor 170 may detect the amount of blue light in the surroundings.

At this time, the control unit 140 compares the amount of blue light detected by the illuminance sensor 170 with a threshold value. As a result of the comparison, if the amount of blue light is greater than the threshold value, the control unit 140 may determine that it is daytime or that natural lighting is present, and may control the maximum variation of the blue light to be reduced.

At this time, the threshold value may be based on 2,500 lux.

Conversely, if the amount of blue light is less than or equal to the threshold value, the control unit 140 may determine that it is nighttime or that natural lighting is absent, and may control the maximum variation of the blue light to be increased.

Accordingly, the mixing ratios of white light, blue light, and red light can be automatically adjusted based on the surrounding illuminance.

The input unit 180 is for allowing the user to manually set the adjustment of the light quantity adjustment unit 120. That is, the white light quantity adjustment variable resistor 121, the blue light quantity adjustment variable resistor 122, and the red light quantity adjustment variable resistor 123 may be varied according to the user's input through the input unit 180.

At this time, the control unit 140 may control the light quantity adjustment unit 120 according to the manual setting via the input unit 180. Accordingly, in addition to the settings based on the myopia prevention program, the user may manually and freely adjust the mixing ratio for each wavelength.

Referring to FIG. 5, it is a graph illustrating time and illuminance related to basic forms of light control in the myopia prevention lighting apparatus according to an exemplary embodiment of the present disclosure. This figure simply shows light control based on illuminance alone under different situations over the passage of time.

As illustrated, the illuminance can be adjusted in various forms according to the passage of time throughout the day.

At this time, the provided light may be a mixture of white light, blue light, and red light, and it is of course possible that light sources of other wavelengths may also be provided.

For example, in the case of A in the graph, the illuminance may increase from midnight and reach 500 lux or more before 6 a.m., and may reach up to 15,000 lux at noon. This case illustrates basic illuminance control, and light control may be performed to provide sufficient illuminance when natural light is not properly available indoors.

In the case of B in the graph, it may represent a situation where natural lighting is not available indoors and illustrates extremely simplified light control.

At this time, during the daytime, that is, between 6 a.m. and 6 p.m., extremely high illuminance may be provided, and during the hours before and after that period, the lowest illuminance close to zero may be provided. This case allows for maximum light exposure during the daytime by providing maximum illuminance when adequate natural lighting is not available.

In the case of C in the graph, when adequate natural lighting is provided indoors, the illuminance may increase linearly between 6 a.m. and 12 p.m. and then decrease after noon, ranging from 500 to 5,000 lux during the daytime between 6 a.m. and 6 p.m. Outside the daytime hours, the illuminance may be controlled to continuously decrease to 500 lux or less as it approaches midnight.

In the case of D in the graph, when adequate natural lighting is provided indoors, it illustrates extreme illuminance control.

As illustrated, the illuminance may be immediately provided at 5,000 lux at 6 a.m. and maintained until 6 p.m. Of course, during the other hours, the lowest illuminance close to zero may be provided.

Referring to FIG. 5, only the illuminance of light has been described with respect to basic general illuminance control and extreme illuminance control. However, various practical cases in which such illuminance control is carried out in relation to the mixing ratios of white light, blue light, red light, and mixed light will be described below.

Hereinafter, examples of wavelength-specific mixing ratios of the myopia prevention lighting apparatus 100 will be described with reference to FIGS. 5 to 11. Here, blue light and red light are described as examples, but it is of course possible that light of other wavelength ranges may be used instead depending on the intended purpose.

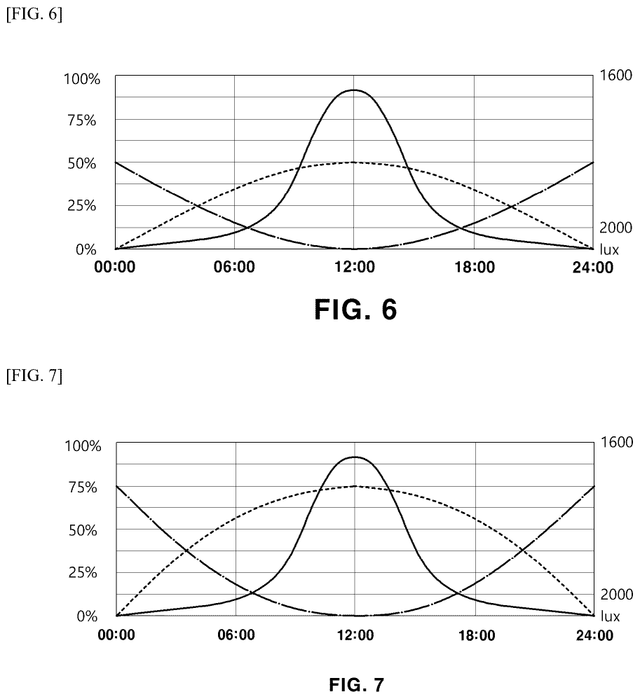

FIG. 5 is a table showing an example of a basic mode of light control in the myopia prevention lighting apparatus according to an exemplary embodiment of the present disclosure, and FIG. 6 is a graph illustrating wavelength composition and changes in illuminance in the basic mode of the myopia prevention lighting apparatus according to an exemplary embodiment of the present disclosure.

FIGS. 5 and 6 illustrate the time-based wavelength mixing ratios in the case where the setting based on the myopia prevention program stored in the program 150 is the basic mode 151. Here, the total mixing ratio of blue light and red light may remain constant over time. That is, the mixing ratios of blue light and red light may form inverse sine waveforms with respect to time.

The mixing ratio of blue light may be higher during the daytime to induce dopamine secretion in the retina and may be lower during the nighttime. For example, the mixing ratio of blue light may be highest at noon and lowest at midnight. However, the magnitude of the mixing ratio of blue light, as well as the peak and minimum timing, are not limited thereto and may be changed depending on the installation location, surrounding illuminance, and user characteristics. At this time, during the daytime-specifically around noon-light of 5,000 lux or more may be provided, and during the nighttime-specifically around midnight-light of 500 lux or less may be provided.

In contrast, the mixing ratio of red light may be higher during the nighttime to induce melatonin secretion and may be lower during the daytime. For example, the mixing ratio of red light may be highest at midnight and lowest at noon. However, the magnitude of the mixing ratio of red light, as well as the peak and minimum timing, are not limited thereto and may be changed depending on the installation location, surrounding illuminance, and user characteristics.

In addition, the total mixing ratio of blue light and red light may be equal to the mixing ratio of white light. That is, the mixing ratio of white light may remain constant over time. As a result, the total mixing ratio of blue light and red light and the mixing ratio of white light may remain constant over time.

For example, as shown in FIG. 5, the total mixing ratio of blue light and red light and the mixing ratio of white light may each be 50%.

In addition, for example, the light may be provided using the aforementioned mixing ratios based on sunrise and sunset times, and at the same time, the illuminance may be adjusted such that 5,000 lux of light is provided from sunrise to sunset and 500 lux of light is provided from sunset to sunrise.

In another example, the peak retinal stimulation period may be from 9 a.m. to 3 p.m., that is, three hours before and after noon, during which the illuminance may reach up to 15,000 lux and remain at or above 5,000 lux. This is because the strongest light stimulus at noon is needed to activate dopamine secretion in the retina, which may result in a myopia prevention effect. It may be most ideal for the period of maximum light exposure at or above 5,000 lux to account for approximately 50% of the daytime, defined as the period from sunrise (6 a.m.) to sunset (6 p.m.).

During the remaining time periods-namely, the intermediate retinal stimulation periods from 6 a.m. to 9 a.m. and from 3 p.m. to 6 p.m.—it may be preferable to provide moderate stimulation with light of 2,000 lux (within a range of 1,000 to 3,000 lux).

At night, the period during which illuminance remains at 500 lux or less may be from sunset to bedtime (6 p.m. to 10 p.m.). After 10 p.m., which corresponds to the sleep period, a lighting level of 250 lux or less may be effective for myopia prevention. In other words, minimal retinal stimulation may suppress dopamine and induce melatonin production.

FIG. 7 is a graph illustrating wavelength composition and changes in illuminance in the case of no natural lighting in the myopia prevention lighting apparatus according to an exemplary embodiment of the present disclosure.

FIG. 7 illustrates the time-based wavelength mixing ratios in the case where the setting based on the myopia prevention program stored in the program 150 corresponds to the case without natural lighting 153. In situations where there is no natural lighting, such as in a fully enclosed indoor environment or where supplementary natural lighting is not available, the maximum variation of blue light having a short wavelength may be set to be greater. This enables effective prevention of myopia.

Here, the total mixing ratio of blue light and red light may remain constant over time. That is, the mixing ratios of blue light and red light may form inverse sine waveforms with respect to time.

As an example of a case without natural lighting, as shown in FIG. 7, the total mixing ratio of blue light and red light may be 75% of the whole, and the mixing ratio of white light may be 25% of the whole.

At this time, the duration during which light is provided at 5,000 lux or more may be longer than that in the basic mode, and the duration during which light is provided at 500 lux or less may be shorter.

Here, as in the case of FIG. 6, the peak retinal stimulation period corresponds to the time span from 9 a.m. to 3 p.m., that is, three hours before and after noon, during which the illuminance may reach up to 15,000 lux and remain at or above 5,000 lux. Of course, as previously described, it may be most ideal for the period of maximum light exposure at or above 5,000 lux to account for approximately 50% of the daytime, defined as the period from sunrise (6 a.m.) to sunset (6 p.m.). In addition, during the nighttime, the period during which illuminance remains at or below 500 lux may likewise be controlled to span from sunset to bedtime, that is, from 6 p.m. to 10 p.m.

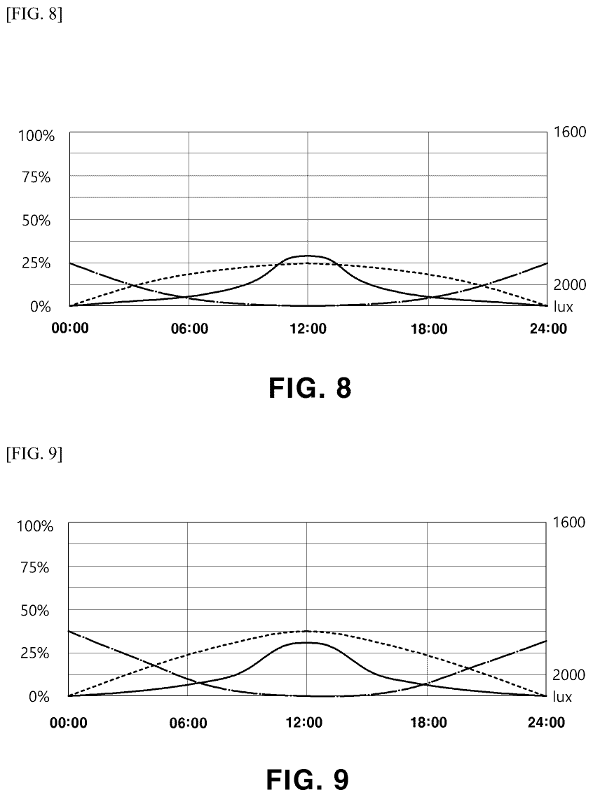

FIG. 8 is a graph illustrating wavelength composition and changes in illuminance in the case of natural lighting in the myopia prevention lighting apparatus according to an exemplary embodiment of the present disclosure.

FIG. 8 illustrates the time-based wavelength mixing ratios in the case where the setting based on the myopia prevention program stored in the program 150 corresponds to the case with natural lighting 152. In cases where supplementary natural lighting is available through windows or the like, the maximum variation of blue light having a short wavelength may be reduced. This may contribute to the prevention of myopia and the reduction of eye fatigue.

Here, the total mixing ratio of blue light and red light may remain constant over time. That is, the mixing ratios of blue light and red light may form inverse sine waveforms with respect to time.

As an example of a case with natural lighting, as shown in FIG. 7, the total mixing ratio of blue light and red light may be 25% of the whole, and the mixing ratio of white light may be 75% of the whole.

At this time, the duration during which light is provided at or above 5,000 lux may be shorter than that in the basic mode, and the duration during which light is provided at or below 500 lux may be longer.

Since natural lighting is provided, the illuminance control may provide up to 5,000 lux, thereby ensuring sufficient illuminance during the daytime in combination with natural light. Of course, it is also possible to provide higher illuminance than that shown in the illuminance graph, as in the previously described embodiments.

FIG. 9 is a graph illustrating wavelength composition in the case of local-area lighting in the myopia prevention lighting apparatus according to an exemplary embodiment of the present disclosure.

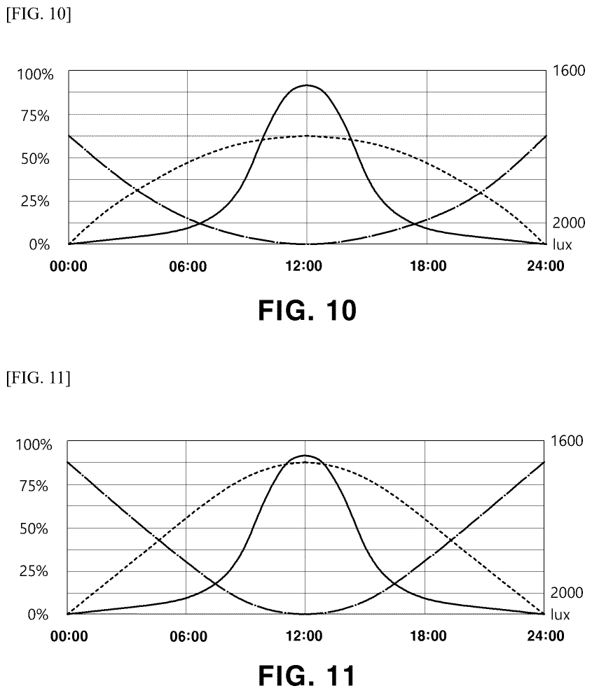

FIG. 10 illustrates the time-based wavelength mixing ratios in the case where the setting based on the myopia prevention program stored in the program 150 corresponds to the local-area lighting 154. In the case of local-area lighting in a room, such as a desk lamp placed on a desk, the maximum variation of blue light and red light may be reduced in consideration of safety. Here, if the contrast between blue light and red light is excessively high in local-area lighting, it may cause eye fatigue.

At this time, the total mixing ratio of blue light and red light may remain constant over time. That is, the mixing ratios of blue light and red light may form inverse sine waveforms with respect to time.

As an example of a case of local-area lighting, as shown in FIG. 8, the total mixing ratio of blue light and red light may be 37.5% of the whole, and the mixing ratio of white light may be 62.5% of the whole.

In terms of illuminance control as well, since local-area lighting is used in a confined space, it is preferable to avoid providing lighting at extremely high illuminance levels such as 15,000 lux, and instead to provide illuminance up to around 5,000 lux.

FIG. 10 is a graph illustrating wavelength composition in the case of full-area lighting in the myopia prevention lighting apparatus according to an exemplary embodiment of the present disclosure.

FIG. 10 illustrates the time-based wavelength mixing ratios in the case where the setting based on the myopia prevention program stored in the program 150 corresponds to the full-area lighting 155. In the case of full-area lighting for a wide space, such as a ceiling-mounted light that illuminates an entire room, the maximum variation of blue light and red light may be increased. This may enhance the efficiency of the lighting.

Here, the total mixing ratio of blue light and red light may remain constant over time. That is, the mixing ratios of blue light and red light may form inverse sine waveforms with respect to time.

As an example of a case of full-area lighting, as shown in FIG. 10, the total mixing ratio of blue light and red light may be 62.5% of the whole, and the mixing ratio of white light may be 37.5% of the whole.

Here, in terms of illuminance control as well, lighting may be provided at an illuminance within the maximum daytime range of 5,000 to 15,000 lux, and during the nighttime for preparing for sleep, lighting may be provided at an illuminance of 500 lux or less as described above.

FIG. 11 is a graph illustrating wavelength composition in the case of a cataract patient using the myopia prevention lighting apparatus according to an exemplary embodiment of the present disclosure.

FIG. 11 illustrates the time-based wavelength mixing ratios in the case where the setting based on the myopia prevention program stored in the program 150 corresponds to the cataracts 156. Due to cataracts, light transmittance is reduced and sensitivity to wavelengths becomes lower. Accordingly, in the case of cataracts, the maximum variation of blue light and red light may be set greater than normal.

Here, the total mixing ratio of blue light and red light may remain constant over time. That is, the mixing ratios of blue light and red light may form inverse sine waveforms with respect to time.

As an example of a case of cataracts, as shown in FIG. 11, the total mixing ratio of blue light and red light may be 87.5% of the whole, and the mixing ratio of white light may be 12.5% of the whole.

In FIGS. 5 to 10, the magnitude of the total mixing ratio of blue light and red light, as well as the peak and minimum timing of the mixing ratios, may of course vary depending on the installation location, surrounding illuminance, and user characteristics.

In addition, at least during the daytime, light of 5,000 lux or more may be provided for a certain period of time, and during the nighttime, light of 500 lux or less may be provided for a certain period of time.

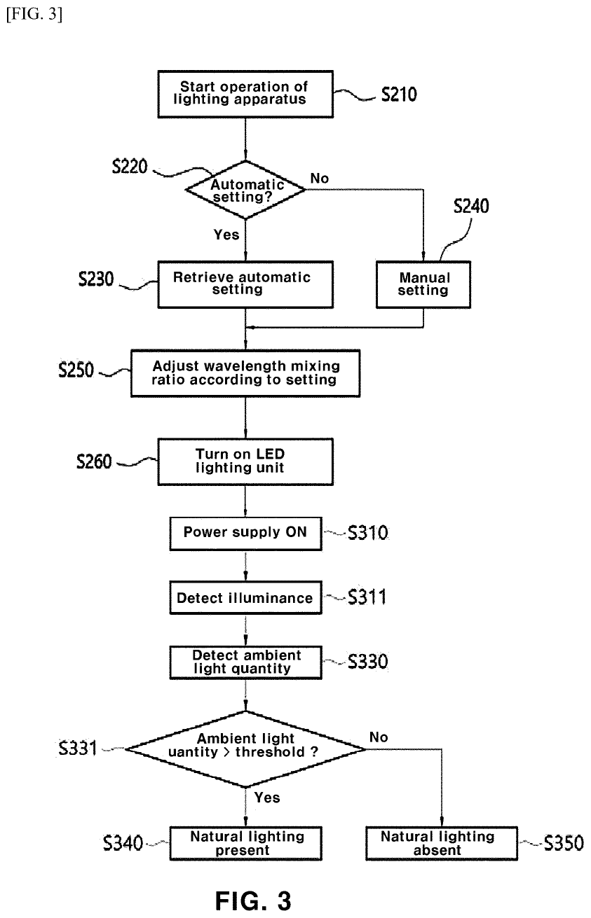

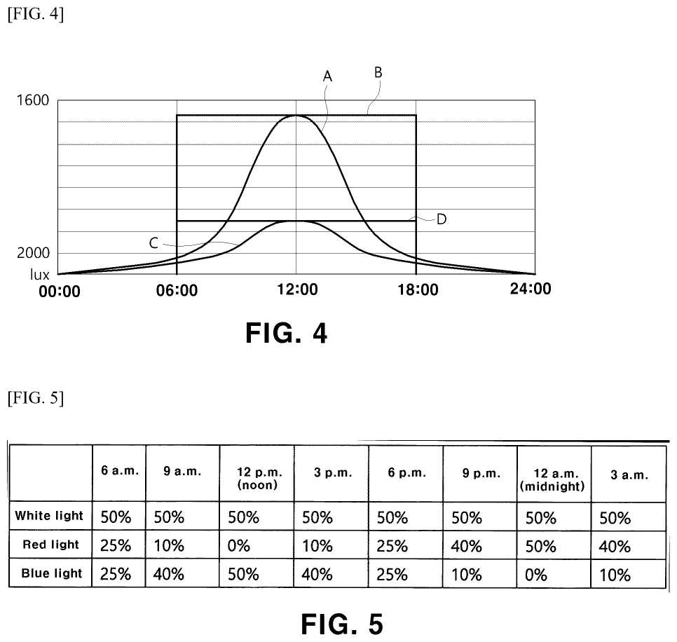

Hereinafter, a control method of the myopia prevention lighting apparatus of the present disclosure will be described with reference to FIG. 3.

FIG. 3 is a flowchart illustrating a control method for the myopia prevention lighting apparatus according to an exemplary embodiment of the present disclosure.

The control method of the myopia prevention lighting apparatus according to an exemplary embodiment of the present disclosure includes: a step S210 of initiating the operation of the lighting apparatus 100; steps S220 to S240 of setting the operation automatically or manually; a step S250 of adjusting the wavelength-specific mixing ratios according to the settings; and a step S260 of turning on the LED lighting unit.

Here, as illustrated in FIG. 11, first, the myopia prevention lighting apparatus 100 initiates its operation (step S210). At this time, the myopia prevention lighting apparatus 100 begins operating as the power is turned on by the user.

Next, the myopia prevention lighting apparatus 100 determines whether the automatic setting is selected (step S220), and if it is the automatic setting, the apparatus retrieves the automatic setting (step S230). That is, the lighting apparatus 100 retrieves the setting of the LED lighting unit 110. Here, the LED lighting unit 110 may include a white LED 111 that emits white light, a first-wavelength LED 112 that emits first-wavelength light, and a second-wavelength LED 113 that emits second-wavelength light having a wavelength range longer than the first wavelength.

Here, the first-wavelength light may be light that promotes the secretion of dopamine. For example, the first-wavelength light may be blue light. Accordingly, the first wavelength may be in the blue light wavelength range. Preferably, the first wavelength may be 450 to 495 nm, and more preferably, it may be 460 nm.

In addition, the second-wavelength light may be light that causes minimal suppression of melatonin. For example, the second-wavelength light may be red light. Accordingly, the second wavelength may be in the red light wavelength range. Preferably, the second wavelength may be 630 to 780 nm, and more preferably, it may be 660 nm.

That is, the LED lighting unit 110 may be composed of mixed light that emits white light for general illumination, blue wavelength ranges optimized for myopia prevention, and red wavelength ranges.

At this time, according to a preprogrammed setting, the mixing ratios of the white light, the blue light, and the red light may be adjusted over time such that, during a certain period in the daytime, the illuminance is 5,000 to 15,000 lux, and during a certain period in the nighttime, the illuminance is 500 lux or less.

At this time, the lighting apparatus 100 may retrieve a setting pre-stored in the program 150.

At this time, if it is determined in step S220 that the manual setting is selected, the lighting apparatus 100 receives a setting input via the input unit 180 to adjust the light quantities of white light, blue light, and red light, and modifies the setting accordingly (step S240).

Next, the lighting apparatus 100 adjusts the mixing ratios of white light, blue light, and red light over time according to the pre-stored automatic setting as in step S230 or the input manual setting as in step S240 (step S250).

At this time, the total mixing ratio of blue light and red light may remain constant over time. That is, the mixing ratios of blue light and red light may form inverse sine waveforms with respect to time.

The mixing ratio of blue light may be higher during the daytime to induce dopamine secretion in the retina and may be lower during the nighttime. For example, the mixing ratio of blue light may be highest at noon and may be provided at 5,000 lux for a certain period of time, and the mixing ratio may be lowest at midnight and may be provided at 500 lux for a certain period of time. However, the magnitude of the mixing ratio of blue light, as well as the peak and minimum timing, are not limited thereto and may be changed depending on the installation location, surrounding illuminance, and user characteristics.

In contrast, the mixing ratio of red light may be higher during the nighttime to induce melatonin secretion and may be lower during the daytime. For example, the mixing ratio of red light may be highest at midnight and lowest at noon. However, the magnitude of the mixing ratio of red light, as well as the peak and minimum timing, are not limited thereto and may be changed depending on the installation location, surrounding illuminance, and user characteristics.

In addition, the total mixing ratio of blue light and red light may be equal to the mixing ratio of white light. That is, the mixing ratio of white light may remain constant over time. As a result, the total mixing ratio of blue light and red light and the mixing ratio of white light may remain constant over time.

In addition, the mixing ratios of white light, blue light, and red light may be adjusted according to the installation location of the lighting apparatus 100, the surrounding illuminance conditions, and the user's health condition such as eye-related diseases. For example, the installation location may be classified as either local-area lighting, such as a desk lamp, or full-area lighting installed on the ceiling. The illuminance condition may be classified based on the presence or absence of natural lighting and automatic setting according to the surrounding illuminance. The eye-related disease may be, for example, cataracts.

At this time, in the case where natural lighting is present or in the case of local-area lighting, the lighting apparatus 100 may adjust the total mixing ratio of blue light and red light to be lower than the mixing ratio of white light.

In addition, in the case of no natural lighting, full-area lighting, or cataracts, the lighting apparatus 100 may adjust the total mixing ratio of blue light and red light to be higher than the mixing ratio of white light.

Here, the total mixing ratio of blue light and red light may be highest in the case of cataracts and lowest in the case of natural lighting. Conversely, the mixing ratio of white light may be lowest in the case of cataracts and highest in the case of natural lighting.

In addition, the total mixing ratio of blue light and red light may be higher in the order of natural lighting absent, full-area lighting, and local-area lighting. Conversely, the mixing ratio of white light may be lower in the order of natural lighting absent, full-area lighting, and local-area lighting.

Next, the lighting apparatus 100 turns on the LED lighting unit 110 according to the wavelength-specific mixing ratios that have been adjusted (step S260).

The automatic control method based on illuminance includes: a step S310 of initiating automatic adjustment based on illuminance; a step S320 of detecting the ambient light quantity; and steps S330 to S350 of performing control according to the presence or absence of natural lighting based on the detected light quantity.

In more detail, first, the lighting apparatus 100 initiates the automatic adjustment operation based on illuminance (step S310). At this time, the lighting apparatus 100 begins the operation as the automatic adjustment operation is selected by the user.

Next, the lighting apparatus 100 detects the ambient light quantity (step S320). At this time, the lighting apparatus 100 may detect the day and night cycle time based on the detected light quantity. For example, the lighting apparatus 100 may detect the amount of blue light in the surroundings.

Next, the lighting apparatus 100 compares the detected amount of blue light with a threshold value (step S330). If the amount of blue light exceeds the threshold value, the lighting apparatus 100 determines that it is daytime or that natural lighting is present, and performs control according to the setting for the case with natural lighting 153 based on the myopia prevention program (step S340). At this time, the lighting apparatus 100 may reduce the maximum variation of blue light.

At this time, the threshold value may be based on a reference value of 2,500 lux.

At this time, if the comparison result in step S330 shows that the amount of blue light is less than or equal to the threshold value, the lighting apparatus 100 determines that it is nighttime or that natural lighting is absent, and performs control according to the setting for the case without natural lighting 152 based on the myopia prevention program (step S350). At this time, the lighting apparatus 100 may increase the maximum variation of blue light.

Such methods may be implemented by the lighting apparatus 100 as shown in FIG. 1, and in particular, may be implemented as a software program that performs such steps, wherein such program may be stored on a computer-readable recording medium or transmitted by a computer data signal combined with a carrier wave in a transmission medium or a communication network. In this case, the computer-readable recording medium may include any kind of recording device in which data readable by a computer system is stored.

Although exemplary embodiments of the present invention have been described, the spirit of the present disclosure is not limited to the embodiments set forth herein. Those of ordinary skill in the art who understand the spirit of the present disclosure may easily propose other embodiments through supplement, change, removal, addition, etc. of elements within the same scope of spirit, but the embodiments will be also within the scope of the present disclosure.

<Description of Symbols>

| | |
|---|---|
| 100: myopia prevention lighting | |
| apparatus | |
| 110: LED lighting unit | 111: white LED |
| 112: blue LED | 113: red LED |
| 120: light quantity adjustment unit | 121: white light quantity |
| | adjustment variable resistor |
| 122: a blue light quantity | 123: a red light quantity |
| adjustment variable resistor 122 | adjustment variable resistor |
| 130: power supply unit | 140: control unit |
| 150: program | 160: timer |
| 170: illuminance sensor | 180: input unit |

What is claimed is:

1. A myopia prevention lighting apparatus comprising:
an LED lighting unit including a white LED that emits white light, a blue LED that emits blue light, and a red LED that emits red light having a wavelength range longer than that of the blue LED;
a power supply unit configured to supply power to the LED lighting unit;
a timer configured to measure time in order to determine day and night cycles;
a light quantity adjustment unit configured to adjust the amounts of the white light, the blue light, and the red light by regulating power supplied from the power supply unit; and
a control unit configured to control the light quantity adjustment unit according to a programmed setting such that the mixing ratios of the white light, the blue light, and the red light are adjusted over time to be equal to or greater than a first illuminance for a certain period during the daytime and equal to or less than a second illuminance for a certain period during the nighttime.

2. The myopia prevention lighting apparatus of claim 1, wherein the first illuminance is in a range of 5,000 to 15,000 lux.

3. The myopia prevention lighting apparatus of claim 1, wherein the second illuminance is in a range of 0 to 500 lux.

4. The myopia prevention lighting apparatus of claim 1,
wherein the total mixing ratio of the blue light and the red light is constant over time,
the mixing ratio of the blue light is higher during the daytime, and
the mixing ratio of the red light is higher during the nighttime.

5. The myopia prevention lighting apparatus of claim 1, wherein the total mixing ratio of the blue light and the red light is equal to the mixing ratio of the white light.

6. The myopia prevention lighting apparatus of claim 1,
wherein the control unit is configured to reduce a maximum variation of the blue light if the amount of light detected at a position irradiated by the LED lighting unit is greater than a threshold value, and to increase the maximum variation of the blue light if the detected light amount is equal to or less than the threshold value.

7. The myopia prevention lighting apparatus of claim 6, wherein the threshold value is 2,500 lux.

8. The myopia prevention lighting apparatus of claim 1,
wherein the control unit is configured to:
control the total mixing ratio of the blue light and the red light to be lower than the mixing ratio of the white light in the case where natural lighting is present or in the case of local-area lighting; and
control the total mixing ratio of the blue light and the red light to be higher than the mixing ratio of the white light in the case where natural lighting is absent, in the case of full-area lighting, or in the case of a cataract patient.

9. A control method for a myopia prevention lighting apparatus, comprising:
retrieving a setting of an LED lighting unit including a white LED that emits white light, a blue LED that emits blue light, and a red LED that emits red light having a wavelength range longer than that of the blue light;
adjusting mixing ratios of the white light, the blue light, and the red light over time according to a preprogrammed setting such that the illuminance is equal to or greater than a first illuminance for a certain period during the daytime and equal to or less than a second illuminance for a certain period during the nighttime; and
turning on the LED lighting unit according to the adjusted mixing ratios.

10. The control method for a myopia prevention lighting apparatus of claim 9, wherein the first illuminance is in a range of 5,000 to 15,000 lux.

11. The control method for a myopia prevention lighting apparatus of claim 9, wherein the second illuminance is in a range of 0 to 500 lux.

12. The control method for a myopia prevention lighting apparatus of claim 9,
wherein the total mixing ratio of the blue light and the red light is constant over time,
the mixing ratio of the blue light is higher during the daytime, and
the mixing ratio of the red light is higher during the nighttime.

13. The control method for a myopia prevention lighting apparatus of claim 9, wherein the total mixing ratio of the blue light and the red light is equal to the mixing ratio of the white light.

14. The control method for a myopia prevention lighting apparatus of claim 9, comprising:
detecting an amount of blue light in the surroundings;
comparing the detected amount of blue light with a threshold value;
reducing a maximum variation of the blue light if the detected amount of blue light is greater than the threshold value; and
increasing the maximum variation of the blue light if the detected amount of blue light is less than or equal to the threshold value.

15. The control method for a myopia prevention lighting apparatus of claim 9,
wherein the adjusting comprises:
adjusting the total mixing ratio of the blue light and the red light to be lower than the mixing ratio of the white light in the case where natural lighting is present or in the case of local-area lighting; and
adjusting the total mixing ratio of the blue light and the red light to be higher than the mixing ratio of the white light in the case where natural lighting is absent, in the case of full-area lighting, or in the case of a cataract condition.
